(19) United States
(12) Patent Application Publication
(10) Pub. No.: US 2010/0210984 A1
(43) Pub. Date: Aug. 19, 2010

(54) Electro-thermal protecting device for heat treatment

(76) Inventor: Chien-Chou Chen, Jiali Town (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 12/379,313

(22) Filed: Feb. 19, 2009

(51) Int. Cl. A61F 5/01

(52) U.S. Cl. 602/2

(57) ABSTRACT

An electro-thermal protecting device includes a protecting body, a heating layer, and a control; the protecting body has a holding part; the control is held in the holding part of the protecting body; the control includes a socket, and several controlling keys used to turn on or turn off the heating layer and set the temperature of the heating layer; the heating layer is joined on an inward side of the protecting body, and includes several high-impedance filaments; a cord is connected to the high-impedance filaments as well as a plug, which can be connected to the socket of the control; the heating layer includes a covering cloth layer wrapped around the high-impedance filaments and the cord thereof; the protecting device is worn on a person's body part so as to protect and warm the body part, and to improve blood circulation and help recovery of the injured body part.

### BACKGROUND OF THE INVENTION

1. Field of the Invention

The present invention relates to an electro-thermal protecting device for heat treatment, more particularly one, which consists of a protecting body, a heating layer on the protecting body, and a control joined on the protecting body and electrically connected to the heating layer to turn on or turn off the heating layer and set the temperature of the heating layer; the protecting device is worn on a person's injured body part to protect and warm the body part, thus improving blood circulation and helping recovery of the body part.

2. Brief Description of the Prior Art

Body protecting band devices are usually used to provide support and compression to sore muscles and sprains, thus preventing the conditions from deteriorating and stopping further swelling of the injured body parts. Waist protectors, shoulder protectors, and knee protectors are among the common body protecting band devices.

Body protecting band devices with heating function are available such as taught in U.S. Pat. No. 5,891,189 (Heating Band Device), and U.S. Pat. No. 5,928,275 (Body Warmer Belt), which can heat the sore/injured body parts besides providing support and compression, thus improving blood circulation and helping recovery of the body parts.

The above-mentioned body protecting band devices include electric plate-shaped heating members on inner sides, and cords, which are connected to the heating members, and extend out of the protecting band devices to be connected to external power supply devices for the electric heating members to be powered through the external power supply devices.

The body protecting band devices are found to have the following drawbacks: first, the plate-shaped heating members have certain stiffness, and therefore can't bend completely according to the curves of the body parts, around which the protecting band devices are wrapped. Consequently, the heating members can't warm the users' body parts efficiently.

Second, the users will have to find a proper place to position the external power supply devices when wearing the protecting band devices. In addition, the cords extend out of the protecting devices, and therefore can easily get damaged or cause other objects to be entangled therein. In other words, the protecting band devices aren't very convenient to use.

Third, the electric heating members of the protecting band devices can't be set or adjusted in temperature according to the users' need.

Therefore, it is a main object of the present invention to provide an improvement on an electro-thermal body protecting device to overcome the above problems.

### SUMMARY OF THE INVENTION

Several different forms of electro-thermal protecting devices are available in accordance with the present invention, which have different shapes so as to serve as a waist protector, shoulder protector, knee protector etc.

An electro-thermal protecting device in accordance with an embodiment of the present invention includes a protecting body, a heating layer, and a control. The protecting body has a holding part. The control is held in the holding part of the protecting body, and it includes a socket, and several controlling keys used to turn on or turn off the heating layer and set the temperature of the heating layer. The heating layer is joined on an inward side of the protecting body, and it includes several high-impedance filaments. A cord is connected to the high-impedance filaments as well as a plug, which is to be connected to the socket. The heating layer includes a covering cloth layer wrapped around the high-impedance filaments and the cord; the protecting device is worn on a person's body part to protect and warm the body part.

An electro-thermal waist protector in accordance with the present invention includes a protecting body, and a heating layer; the protecting body has a rectangular section, and tying bands extending from two ends of the rectangular section; one of the tying bands has a joining piece thereon.

An electro-thermal shoulder protector of the invention includes a protecting body, and a heating layer; the protecting body has a recessed portion to be positioned on a person's shoulder; the protecting body has tying bands extending from two ends of a lower portion of the recessed portion, a fastening band joined to one end of an upper portion of the recessed portion, and a fastening piece on the other end of the upper portion of the recessed portion. The heat layer consists of two curved plates, which are joined together to be in the shape of a polygonal plate, and held in the recessed portion of the protecting body.

An electro-thermal knee protector of the invention includes a protecting body, and a heating layer; the protecting body includes a semispherical portion to cover a person's knee, and a tying plate part extending from two sides of the semispherical portion. The heat layer is substantially in the shape of a round plate with several apertures, and it is positioned in the semispherical portion of the protecting body.

### BRIEF DESCRIPTION OF THE DRAWINGS

The present invention will be better understood by referring to the accompanying drawings, wherein:

FIG. 1 is a perspective view of the present invention,

FIG. 2 is an exploded perspective view of a first preferred embodiment of the present invention,

FIG. 3 is a sectional view of the present invention,

FIG. 4 is an exploded perspective view of a second preferred embodiment of the present invention,

FIG. 5 is an exploded perspective view of a third preferred embodiment of the present invention, and

FIG. 6 is a circuit diagram of the present invention.

### DETAILED DESCRIPTION OF THE PREFERRED EMBODIMENTS

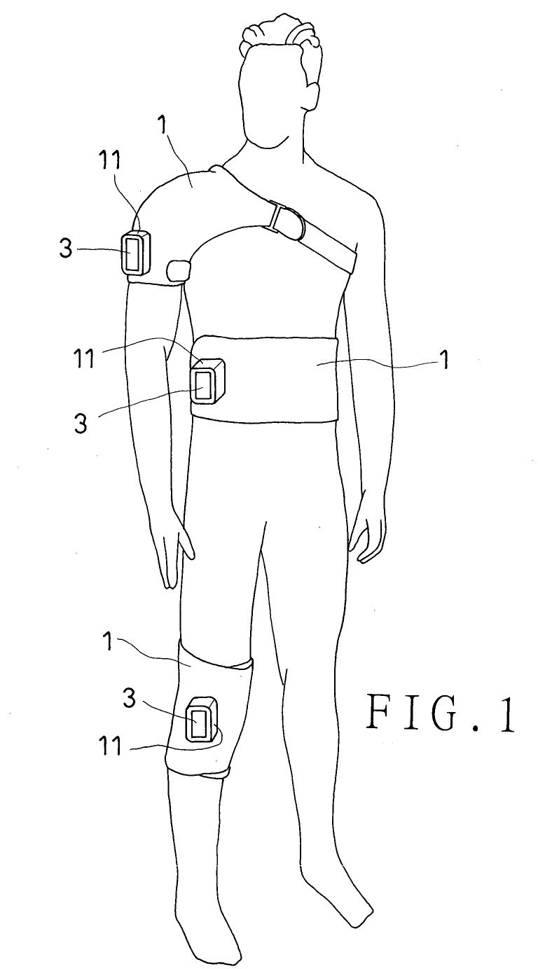

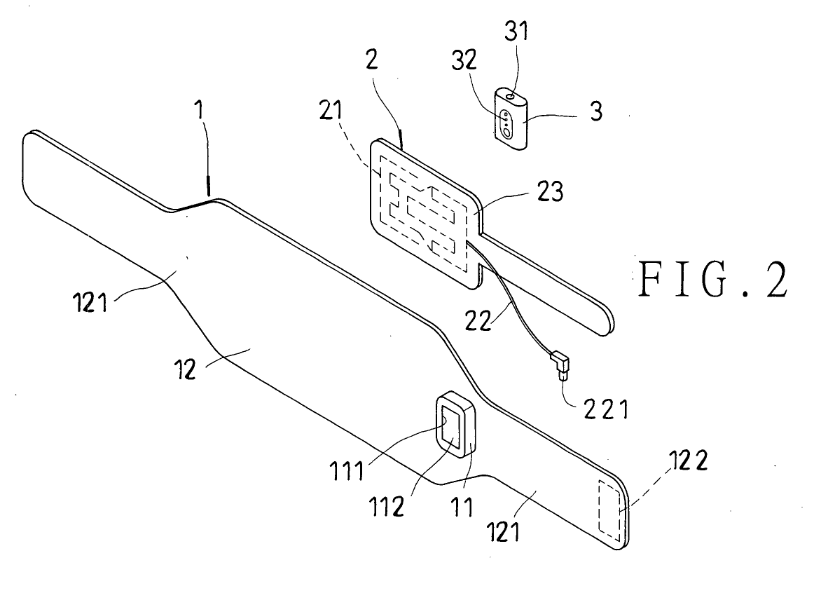

Shown in FIGS. 1 and 2 is a first preferred embodiment of an electro-thermal protecting device for heat treatment in the present invention, which consists of a protecting body 1, a heating layer 2, a control 3, and a battery charger 4.

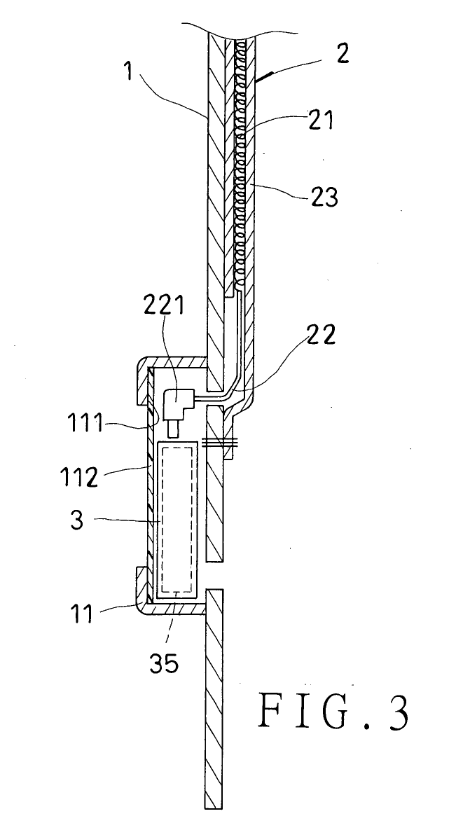

The protecting body 1 has a holding part 11, and the control 3 is held in the holding part 11; the holding part 11 has an opening 111, and a transparent film 112 is positioned over the opening 111, as shown in FIG. 3; thus, the user can see the panel of the control 3, which has a display unit (not shown) to indicate the temperature of the heating layer 2.

The first referred embodiment is formed in such a manner as to serve as a waist protector; the protecting body 1 includes a rectangular section 12, and tying bands 121 extending from two ends of the rectangular section 12; one of the tying bands 121 has a joining piece 122 thereon.

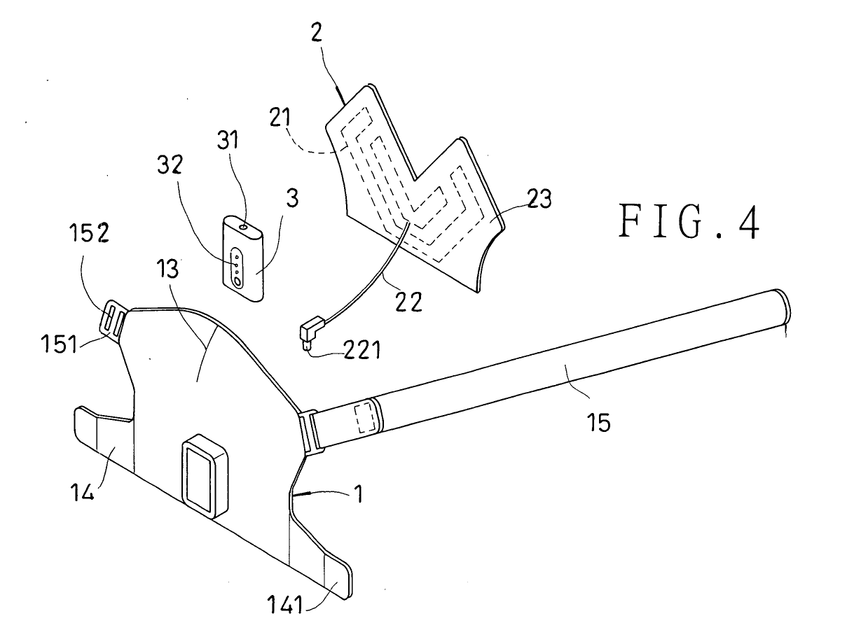

Shown in FIG. 4 is a second preferred embodiment of an electro-thermal protecting device of the present invention, which is formed in such a manner as to serve as a shoulder protector; the protecting body 1 has a recessed portion 13 to be positioned on a person's shoulder. The protecting body 1 further has tying bands 14 extending from two ends of a lower portion of the recessed portion 13; the tying bands 14 each have a joining piece 141 on a free end thereof. The protecting body 1 has a fastening band 15 joined to a first end of an upper portion of the recessed portion 13, and a fastening piece 151 on a second end of the upper portion of the recessed portion 13; the fastening piece 151 has a fastening hole 152 for a tail end of the fastening band-15 to be passed through and fixed.

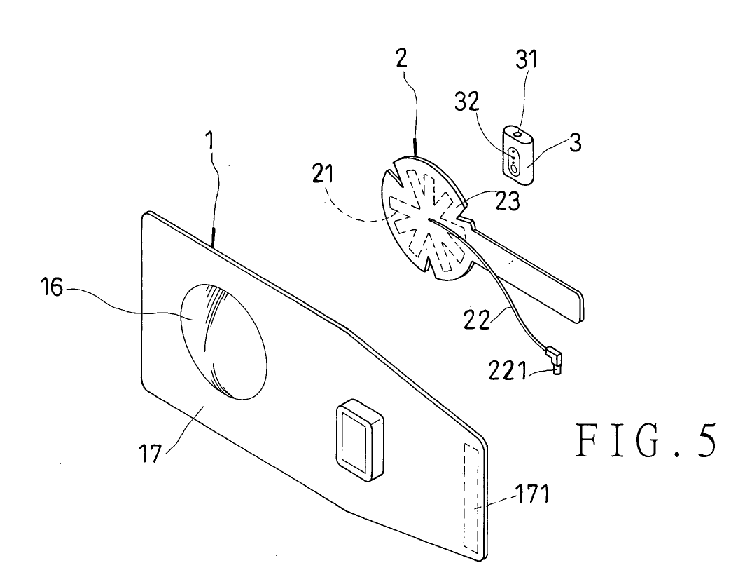

Shown in FIG. 5 is a third preferred embodiment of an electro-thermal protecting device of the present invention, which is formed in such a manner as to serve as a knee protector; the protecting body 1 includes a semispherical portion 16 to cover a person's knee; the protecting body 1 further includes a tying plate part 17 extending from two sides of the semispherical portion 16; the tying plate part 17 has a joining piece 171 on one end thereof.

The heating layer 2 is joined on an inward side of the protecting body 1; the heating layer 2 includes high-impedance filaments 21, which can be carbon fiber filaments; the heating layer 2 includes an electricity conducting cord 22, which is connected to the high-impedance filaments 21; the heating layer 2 further includes covering cloth layers 23, which are wrapped around the high-impedance filaments 21 and the electricity conducting cord 22. The cord 22 extends out of the covering cloth layers 23, and has a plug 221 connected thereto.

Referring to FIG. 2 again, the heat layer 2 is in the shape of a rectangular plate according to the first preferred embodiment (a waist protector), and it is positioned on the rectangular section 12 of the protecting body 1.

Referring to FIG. 4 again, the heat layer 2 consists of two curved plates, which are joined together to be in the shape of a polygonal plate, according to the second preferred embodiment (a shoulder protector); the polygonal heating layer 2 is held in the recessed portion 13 of the protecting body 1.

Referring to FIG. 5 again, the heat layer 2 is substantially in the shape of a round plate with several apertures, according to the third preferred embodiment (a knee protector), and it is positioned in the semispherical portion 16 of the protecting body 1.

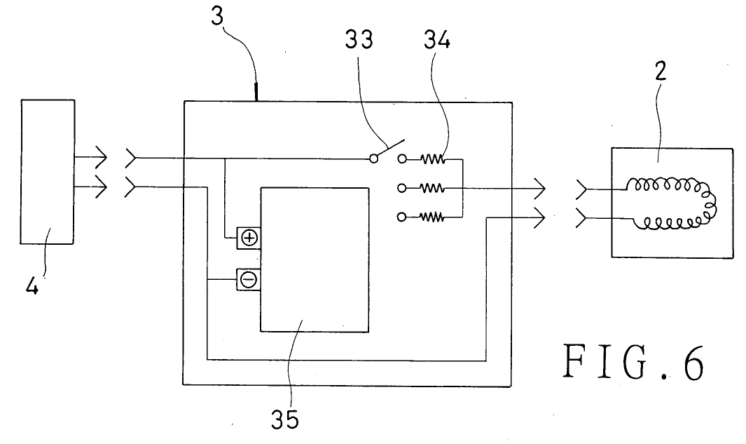

Referring to FIG. 6, the control 3 has a socket 31 on one end thereof for connection with the plug 221 joined on the electricity conducting cord 22 of the heating layer 2. The control 3 includes a temperature controlling switch 33, and several temperature controlling elements 34, and it has several controlling keys 32, which are connected to the temperature controlling switch 33 and the temperature controlling elements 34; the temperature controlling elements 34 can be temperature controlling resistors. The control 3 further includes a display unit to indicate the temperature. The control 3 contains batteries 35 therein, which are rechargeable batteries; a battery charger 4 can be connected to the control 3 to charge the batteries 35.

Referring to FIG. 1, the above-mentioned three different forms of electro-thermal protecting devices are worn on a person's injured shoulder, waist and knee respectively; the heating layer 2 will start giving out heat to warm the user's injured body part like a hot compress after the control 3 is turned on. The temperature of the heating layer 2 is set through the control 3. Referring to FIGS. 1 and 2, if the user has an injured waist, a protecting device in accordance with the first embodiment should be fastened around the user's injured waist with the rectangular section 12 of the protecting body 1 covering a large portion of the waist, and with the tying bands 1211 being joined together; heat given out from the heating layer 2 will pass through the protecting body 1 to warm the injured waist. The high-impedance filaments 21 of the heating layer 2 are very soft, and therefore will change shape easily according to the curves of the user's waist; in other words, the heating layer 2 will be closely in contact with the injured part of the user's waist so as to improve blood circulation and help recovery of the injured body part.

Referring to FIGS. 1 and 4, if the user has an injured shoulder, a protecting device in accordance with the second embodiment should be fastened over the injured shoulder with the recessed portion 13 of the protecting body 1 being positioned on the shoulder, the tying bands 14 being positioned around an upper portion of the user's arm and joined together with the joining pieces 141, and with the fastening band 15 being passed over other side of the user's chest and joined to the fastening hole 152 of the fastening piece 151; thus, the heating layer 2 will give out heat to warm the injured shoulder.

Referring to FIGS. 1 and 5, if the user has an injured knee, a protecting device in accordance with the third embodiment should be fastened around the injured knee with the semispherical portion 16 of the protecting body 1 covering the knee, and the tying plate parts 17 being positioned around the knee and joined together with the joining pieces 171; thus, the heating layer 2 will give out heat to warm the injured knee.

Referring to FIG. 3, the user can see the controlling keys 32 of the control 3 and the set temperature value shown on the display unit through the transparent film 112 secured over the opening 111 of the holding part 11. Therefore, he/she can operate the controlling keys 32 to adjust the temperature of the heating layer 2 according to his/her need.

From the above description, it can be seen that the present invention has the following advantages:

1. Three different forms of electro-thermal protecting devices are available according to the present invention, which are waist, shoulder and knee protectors, and can be worn on a person's waist, shoulder and knee respectively to give out heat for heat treatment, thus improving the blood circulation, and helping recovery of the body parts if the body parts are injured.

2. The electro-thermal waist protector of the present invention has the protecting body with a rectangular section, and therefore can contact the wearer's waist in such a manner as to warm the waist efficiently. The electro-thermal shoulder protector of the invention has the protecting body with a recessed portion, and therefore can contact the wearer's shoulder in such a manner as to warm the shoulder efficiently. The electro-thermal knee protector of the invention has the protecting body with a semispherical portion, and therefore can come closely in contact with the wearer's knee to produce desired warming effect.

3. The high-impedance filaments of the heating layer are very soft, and therefore can change shape easily according to the curves of the user's body. Consequently, the heating layer will be closely in contact with the user's injured body part to improve blood circulation and help recovery of the injured body part.

4. The control of the present invention is hidden in the holding part of the protecting body while the heating layer is joined on the inward side of the protecting body with the cord being connected to the control; the user won't have to find a proper position to place the control, and the cord is properly protected to not get damaged or cause other objects to get entangled therein.

5. The temperature of the heating layer of the electro-thermal protecting device can be set and adjusted through the controlling keys of the control. Therefore, the present invention is convenient to use.

6. The holding part of the protecting body has an opening, and a transparent film is secured over the opening. Therefore, the user can see the temperature display unit of the control, and operate the controlling keys to adjust the temperature of the heating layer.

What is claimed is:

1. An electro-thermal protecting device for heat treatment, comprising
a protecting body; the protecting body having a holding part; the holding part having an opening thereon; the holding part having a transparent film positioned over the opening thereof;
a heating layer joined on an inward side of the protecting body; the heating layer including plural high-impedance filaments; the heating layer including an electricity conducting cord connected to the high-impedance filaments thereof; the heating layer including plural covering cloth layers wrapped around the high-impedance filaments and the electricity conducting cord; the electricity conducting cord extending out of the covering cloth layers; the electricity conducting cord having a plug joined thereon; and
a control held in the holding part of the protecting body; the control having a socket on one end thereof for connection with the plug joined on the electricity conducting cord of the heating layer; the control including a temperature controlling switch, and plural temperature controlling elements; the control having plural controlling keys connected to the temperature controlling switch and the temperature controlling elements thereof; the control containing plural batteries therein.

2. The electro-thermal protecting device for heat treatment as claimed in claim 1, wherein the protecting body is a waist protector, and includes a rectangular section, and tying bands extending from two ends of the rectangular section; one of the tying bands having a joining piece on one end thereof; the heat layer being in a shape of a rectangular plate and positioned on the rectangular section of the protecting body.

3. The electro-thermal protecting device for heat treatment as claimed in claim 1, wherein the protecting body is a shoulder protector, and has a recessed portion to contact a wearer's shoulder;
the protecting body having tying bands extending from two ends of a lower portion of the recessed portion; the tying bands having a joining piece on one end thereof; the protecting body having a fastening band joined to a first end of an upper portion of the recessed portion; the protecting body having a fastening piece on other end of the upper portion of the recessed portion thereof; the fastening piece having a fastening hole thereon for a tail end of the fastening band to be passed through and fixed;
the heat layer including two curved plates; the curved plates of the heating layer being joined together to be in a shape of a polygonal plate; the heating layer being held in the recessed portion of the protecting body.

4. The electro-thermal protecting device for heat treatment as claimed in claim 1, wherein the protecting body is a knee protector, and includes a semispherical portion to contact a person's knee;
the protecting body including a tying plate part extending from two sides of the semispherical portion thereof; the tying plate part having a joining piece on one end thereof.
the heat layer being substantially in a shape of a round plate with plural apertures; the heating layer being positioned in the semispherical portion of the protecting body.

5. The electro-thermal protecting device for heat treatment as claimed in claim 1, wherein the high-impedance filaments are carbon fiber filaments.

6. The electro-thermal protecting device for heat treatment as claimed in claim 1, wherein the control includes a temperature display unit.

7. The electro-thermal protecting device for heat treatment as claimed in claim 1, wherein the batteries are rechargeable ones.

8. The electro-thermal protecting device for heat treatment as claimed in claim 7 further including a battery charger to charge the rechargeable batteries.
